(19) United States
(12) Patent Application Publication
(10) Pub. No.: US 2008/0242997 A1
(43) Pub. Date: Oct. 2, 2008

(54) METHOD AND APPARATUS FOR CLASSIFYING GASEOUS AND NON-GASEOUS OBJECTS

(75) Inventors: John E. LYNCH, Williamsburg, VA (US); John K. Lynch, Fredricksburg, VA (US)

Correspondence Address: NIXON & VANDERHYE, PC, 901 NORTH GLEBE ROAD, 11TH FLOOR, ARLINGTON, VA, 22203, US

(73) Assignee: Luna Innovations Incorporated, Roanoke, VA (US)

(21) Appl. No.: 12/053,289

(22) Filed: Mar. 21, 2008

Related U.S. Application Data
(60) Provisional application No. 60/907,209, filed on Mar. 26, 2007.

(51) Int. Cl. A61B 8/06

(52) U.S. Cl. 600/455

(57) ABSTRACT

Ultrasonic pulse echo object classification is described. A broadband ultrasound transducer transmits a broadband ultrasound pulse towards the object and detecting an associated ultra sound echo of that pulse from the object. An ultrasound receiver receives the detected echo signal. A signal processor, coupled to the ultrasound receivers determines and analyzes a time duration parameter and a frequency parameter (and possibly one or more other parameters like amplitude and/or phase) of the detected echo signal and classifies the object as a solid or liquid or as gaseous based on the parameters of the detected echo signal.

### RELATED APPLICATIONS

This application claims priority from U.S. provisional patent application Ser. No. 60/907,209, filed on Mar. 26, 2007, the contents of which are incorporated herein by reference. This application is related to commonly-assigned U.S. patent application Ser. No. 11/429,432, filed on May 8, 2006, the contents of which are also incorporated herein by reference.

### TECHNICAL FIELD

The technical field relates to detecting and classifying objects using ultrasound technology. One non-limiting application is to detect and classify different types of emboli in the bloodstream.

### BACKGROUND

Embolic particles carried by the bloodstream can causes strokes and other circulatory disorders. During surgery emboli may occur when clots form in the blood, air enters into the bloodstream, or tissue fragments break loose or become dislodged. The blood carries the emboli into increasingly smaller arteries until they become lodged and obstruct the flow of blood. The amount of damage that results depends on the size of the emboli, the point in which it lodges in the blood flow, the amount of blood leaking around the emboli, and how blood is supplied by collateral paths around the obstruction. The resulting functional deficit depends in part on the composition of the emboli. For example, air may be reabsorbed in a short time, clots may dissolve, (particularly if blood-thinning drugs are present), while particles composed of plaque and body tissue may not dissolve at all. Therefore, it is important to have non-invasive instrumentation that can accurately detect the presence of emboli, determine their composition, and estimate their size so that appropriate medical management decisions can be made.

Instrumentation for detecting and classifying emboli based on ultrasound is described in U.S. Pat. No. 5,441,051, the disclosure of which is incorporated here by reference. When an emboli passes through an ultrasound beam, the change in acoustic reflectivity causes a reflection which can be detected by an ultrasound receiver. In the '051 patent, the number of embolic events can be counted by monitoring the number of reflected echoes that exceed a predetermined threshold. The '051 patent also describes a method to characterize emboli by composition and size so that an embolus may be classified for example as a gas or a fat particle based on a polarity of the echo signal for each embolus.

Before moving objects like emboli can be accurately counted and classified, reflected signals from the moving object need to be processed to eliminate reflections from stationary objects that are of less interest. In the blood scanning application, these stationary objects include the blood vessel walls and surrounding tissue. The reflections from surrounding tissue are generally stronger than those from the flowing blood and from the emboli. The strong reflections from stationary objects may be reduced using a moving object indicator (MOI). An MOI temporarily stores one line of echo data and subtracts it from a subsequent line of echo data. Differencing two lines of echo data substantially cancels the stationary object signals leaving the signal reflected from the moving objects, e.g., from the blood flow and the emboli contained therein.

The noise performance of an ultrasonic moving object indicator is a significant issue. One way of improving noise performance is to average multiple lines in such a way that the signal-to-noise ratio is improved. In that case, differences are determined between the averages. The signal-to-noise ratio improves by a factor of the square root of the number of lines averaged when the noise is incoherent and the reflected signal is coherent. Averaging multiple lines results in a waveform that responds slowly to changes. The averaged waveform does not change significantly even when a moving object, e.g., an embolus, passes through the ultrasound beam. The differencing, however, produces a large value when the moving object is present in the ultrasound beam. In addition, the averaging “filter” still leaves significant background noise artifacts.

Commonly-assigned U.S. application Ser. No. 11/429,432, filed on May 8, 2006, the contents of which are incorporated here by reference, describes an improved performance ultrasonic moving object indicator. The improved signal-to-noise performance of this MOI results in more accurate detection of the objects and their ultrasonic echo signatures. This echo signature may be used to classify the composition of the embolus. Other embolus classification techniques have been described in the following, the contents of which are incorporated here by reference:
1. Ajzan et al. “Quantification of Fat Mobilization in Patients Undergoing Coronary Artery Revascularization Using Off-pump and On-pump Techniques.” JECT. 2006; 38:116-121.
2. Xu et al. “An Automated Feature Extraction And Emboli Detection System Based On The Pca And Fuzzy Sets.” Computers In Biology And Medicine, Available online 27 Oct. 2006.
3. U.S. Pat. Nos. 5,348,015; 6,616,611; 6,547,736; 6,524,249; and 6,196,972.
4. Devuyst et al. “Automatic Classification Of Hits Into Artifacts Or Solid Or Gaseous Emboli By A Wavelet Representation Combined With Dual-Gate TCD,” Stroke (2001); 32;2803-2809.
5. Smith et al. “Time Domain Analysis Of Embolic Signals Can Be Used In Place Of High-Resolution Wigner Analysis When Classifying Gaseous And Particulate Emboli.” Ultrasound in Med. & Biol., Vol. 24, No. 7, pp. 989-993, 1998.
6. Cowe et al. “RF signals provide additional information on embolic events recorded during TCD monitoring.” Ultrasound in Medicine & Biology, May 2005; 31(5):613-23.
7. El-Brawany et al. Microemboli Detection Using Ultrasound Backscatter. Ultrasound In Medicine & Biology, Volume 28, Issues 11-12, November-December 2002, Pages 1439-1446.

Most of the documents in the above list are based on variations of Doppler ultrasound technique. In most cases, the time waveform of the down-converted Doppler signal is analyzed to distinguish solid from gaseous emboli (and from artifacts). This Doppler signal is highly variable with blood velocity, transducer beam shape, the position of the embolus within the ultrasound beam, and the composition of the embolus. For example, the amplitude of the time waveform of the Doppler signal may be affected by the position of the emboli within the ultrasound beam (with emboli near the center of the beam producing larger amplitude signals than emboli near the edges of the beam) as well as the size and composition of the emboli. Similarly, the duration of the time waveform of the Doppler signal may be affected by the blood velocity, as well as the position of the emboli within the ultrasound beam (with emboli near the ultrasound beam focus producing a shorter duration signal than emboli away from the focus). The interdependence of these variables makes it difficult to extract reliable information from the time waveform concerning the size and composition of the emboli.

Advanced multi-gate techniques and time-frequency analysis (such as wavelets) have been employed in many of the listed references, but these have only brought incremental improvements to a fundamentally error-prone technique. The Cowe et al reference attempts to classify the RF return of a transcranial Doppler system rather than the down-shifted Doppler signal. However, the long tone burst employed in a Doppler system tends to blur subtle effects in echo ring-down time and frequency-dependent backscattering.

The El-Brawany et al reference describes a backscatter approach that employs broadband ultrasonic signals, but treats the ultrasonic echo as a chaotic signal in which the discrimination of echoes from solids and gases is performed using a purely mathematical model. This approach is easily confounded by changes in experimental test conditions, and is difficult to transfer from the laboratory to clinical use. There is no indication that it has ever been used outside the laboratory.

U.S. Pat. No. 5,441,051 briefly mentions numerous measurement methodologies such as the Fast Fourier Transform (FFT), deconvolution, matched filters, neural networks, and artificial intelligence. But specific details of how these techniques might be used in classification are not provided. Some detail is provided on how to use the phase/polarity of an echo to discriminate emboli since emboli are more dense than the surrounding fluid and said to have an inverted phase from emboli less dense that the surrounding fluid. However, this approach only applies to specular reflections, not to Rayleigh scattering from small particles, which is more relevant to emboli detection. Even in the case of specular reflection, this phase measurement misclassifies oils, which are less dense than blood and water, as gaseous emboli.

### SUMMARY

The technology in this application provides an ultrasonic pulse echo apparatus for classifying an object that overcomes deficiencies with the approaches identified in the background. A broadband ultrasound transducer transmits a broadband ultrasound pulse towards the object and detects an associated ultrasound echo of that pulse from the object. An ultrasound receiver receives the detected echo signal. A signal processor, coupled to the ultrasound receiver, determines and analyzes a time duration parameter and a frequency parameter of the detected echo signal and classifies the object as (1) a solid or liquid or (2) gaseous based on the time duration parameter and the frequency parameter of the detected echo signal. For example, the object may be classified as a solid or liquid when the time duration parameter exceeds a predetermined time duration value and the frequency parameter exceeds a predetermined frequency value. Otherwise, the object is classified as gaseous.

In a preferred but non-limiting embodiment, a computer-implemented statistical classification algorithm determines a classification threshold based on the frequency and time duration parameters of the detected echo signal. The statistical classification algorithm performs, for example, a logistic regression that combines the frequency and time duration parameters of the detected echo signal. Higher values of the frequency and time duration parameters produce a statistical result indicating a higher probability that the object is a solid or liquid rather than gaseous. Other statistical analyses could include other methods, such as but not limited to discriminant analysis, recursive partitioning, etc.

In another example embodiment, an amplitude parameter and a phase parameter of the detected echo signal are also analyzed. The object may then be classified as a solid or liquid or as gaseous based on the time duration parameter, the frequency parameter, and one or both of the amplitude parameter and the phase parameter of the detected echo signal.

The technology is effective for classifying both stationary objects and moving objects. In one advantageous medical application, the object may be an embolus in a blood stream, and the embolus may be classified as a gas bubble, a clot, or a solid particle. Moreover, the technology has other useful applications such as determining a density of an object.

Preferably, the broadband transducer has a percent bandwidth of at least 50% of a center frequency of the transducer. In one non-limiting example, the broadband transducer is a piezoelectric composite transducer and has a bandwidth frequency response range between approximately 1 MHz and 10 MHz.

The technology may be embodied as an apparatus, method, and/or a computer program product which includes a computer program embodied on a computer-readable medium for controlling a computer.

### BRIEF DESCRIPTION OF THE DRAWINGS

FIG. 1 is a function block diagram illustrating one non-limiting example of an ultrasonic detection apparatus;

FIG. 2 is a flow chart diagram illustrating non-limiting example steps for classifying an object using a detected ultrasound echo;

FIG. 3 illustrates an example echo waveform in the time and frequency domains;

FIG. 4 illustrates an example echo waveform in the time domain identifying four echo signal parameters;

FIG. 5A illustrates echo waveforms in the time domain for a test echo, a gas bubble echo, and a solid sphere echo;

FIG. 5A illustrates echo waveforms in the frequency domain for a test echo, a gas bubble echo, and a solid sphere echo;

FIG. 6 illustrates an example echo waveform (signature) used in a laboratory test;

FIG. 7 illustrates an envelope of the example echo waveform shown in FIG. 6;

FIG. 8 illustrates a phase plot of the example echo waveform shown in FIG. 6; and

FIG. 9 illustrates a receiver operator characteristic curve showing sensitivity and specificity of example classification test results.

### DETAILED DESCRIPTION

In the following description, for purposes of explanation and non-limitation, specific details are set forth in order to provide an understanding of the described technology It will be apparent to one skilled in the art that other embodiments may be practiced apart from the specific details disclosed below. In other instances, detailed descriptions of well-known methods, devices, techniques, etc. are omitted so as not to obscure the description with unnecessary detail. Individual function blocks are shown in the figures. Those skilled in the art will appreciate that the functions of those blocks may be implemented using individual hardware circuits, using software programs and data in conjunction with a suitably programmed microprocessor or general purpose computer, using applications specific integrated circuitry (ASIC), field programmable gate arrays, one or more digital signal processors (DSPs), etc.

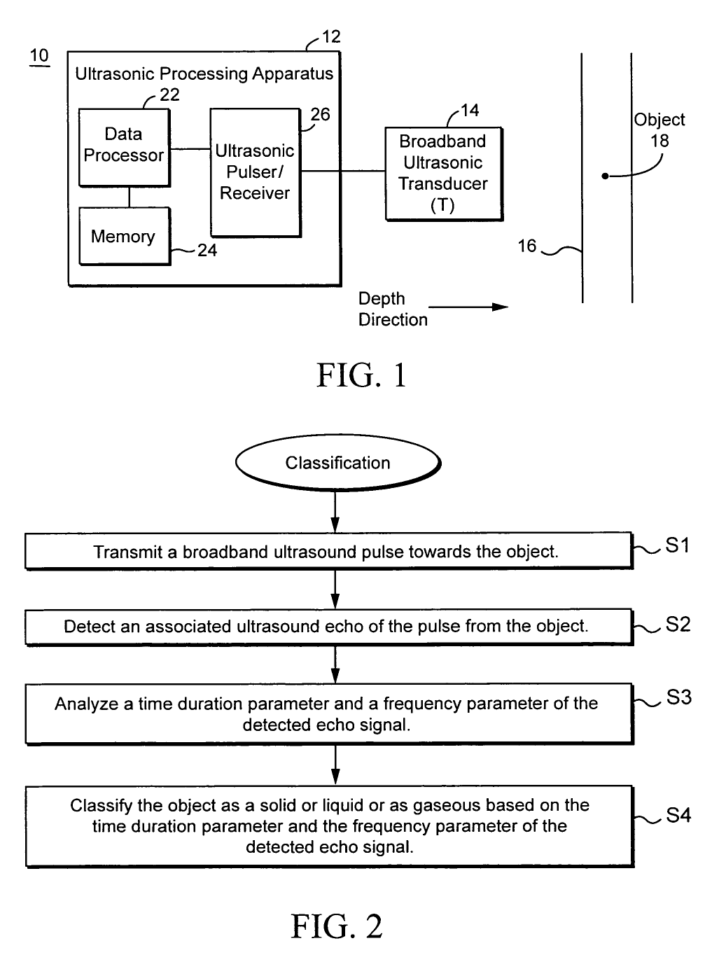

FIG. 1 shows a non-limiting example embodiment of an object classification system which is indicated by the numeral 10. For purposes of explanation only, and not limitation, the object classification apparatus 10 is sometimes described in the context of an emboli classification application. Of course, this technology may be used with other applications.

The object classification system 10 includes an ultrasonic processing apparatus 12 that controls an ultrasound transducer 14 positioned so that a stationary object 18 located near the ultrasound transducer 14 or a moving object 18 passes by the ultrasound transducer 14, ultrasonic pulses impinge on the object resulting in one or more reflected echoes that are detected by the ultrasound transducer 14. The ultrasonic processing apparatus 12 includes a data processor 22 coupled to memory 24 and to an ultrasonic pulser/receiver 26. Although not necessary, the ultrasonic processing apparatus 12 may be similar to that described in commonly-assigned U.S. application Ser. No. 11/429,432, filed on May 8, 2006, which describes how to improve the performance of an ultrasonic moving object indicator. Another example ultrasonic processing apparatus is the Emboli Detection And Classification EDAC® Quantifier from Luna Innovations Incorporated

A tube or vessel 16 with close and far walls is insonified by the ultrasonic pulses. In the example emboli classification application, the tube corresponds to blood vessel walls or walls of other blood transport conduit, and the object 18 corresponds to an embolus. The term “depth” corresponds to the perpendicular direction away from the ultrasound transducer 14 towards the object.

The ultrasound transducer 14 transmits ultrasound pulses and receives one or more ultrasound echoes or reflections from the object. As one non-limiting example, the transducer 14 may be a piezoelectric transducer, preferably a PZT composite having a quarter wave impedance matching layer to increase the coupling of sound from the transducer 14 into the object. The ultrasonic pulser 26 also preferably (but not necessarily) applies fast-rise time step pulses to the transducer 14 which is converted by the transducer 14 into ultrasound signals that reflect off the object being scanned. One non-limiting example drive pulse has a voltage over 100 volts and a rise time on the order of 15 nanoseconds.

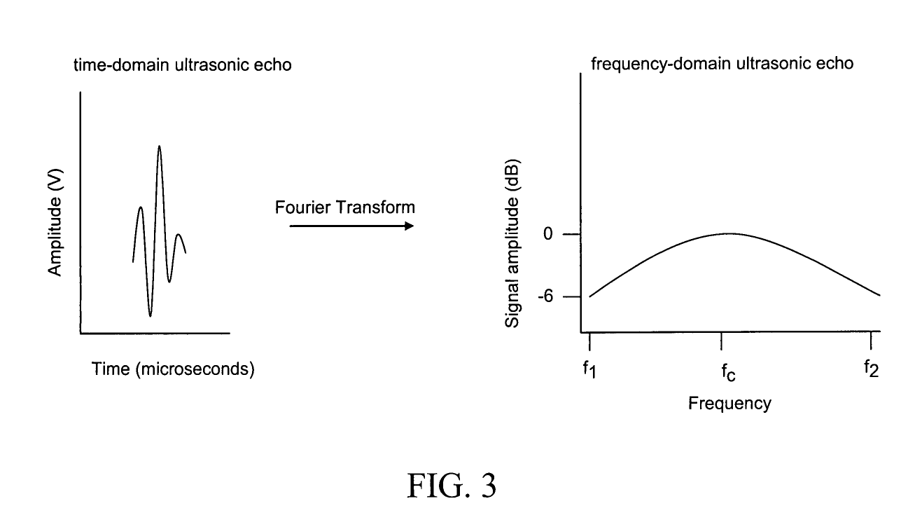

Ultrasonic reflections or echoes return to the transducer 14 which converts the reflected acoustic energy into corresponding electronic echo signals. The transducer 14 preferably has a broad bandwidth so that, among other things, it can detect frequency shifts in the return echo and differences in echo “ring-down” time. FIG. 3 is helpful in understanding why a broadband transducer is preferred. On the left side of FIG. 3, an example ultrasonic echo in the time-domain is shown. That signal is transformed into the frequency domain by the Fourier transform. Reflection of the ultrasound pulse shifts the frequency of the acoustic signal so that the echo must be detected at a frequency that is substantially different than the frequency of the transmitted pulse. The curve in the frequency plot of FIG. 3 illustrates a frequency band, i.e., a bandwidth, over which a transducer must be sensitive. The bandwidth is understood to be the range of frequencies that can be detected −6 dBs down from the center frequency f_{c}. Preferably, the broadband transducer has a percent bandwidth of at least 50% of the center frequency of the broadband transducer. In one non-limiting example, the broadband transducer is a composite type piezoelectric transducer and has a bandwidth frequency range between approximately 1 MHz and 10 MHz. A plurality of ultrasound transducers may be arranged in an array and operated sequentially to produce adjacent beams that collectively cover larger areas.

The ultrasonic receiver 26 preferably includes amplification, time gain compensation, filtering, and analog-to-digital conversion. The ultrasonic receiver 26 amplifies the electrical echoes from the transducer 14 to a level suitable for analyzing and processing. Time gain compensation increases the gain with time to compensate for the acoustic attenuation experienced as the ultrasound pulse travels deeper in the depth direction shown in FIG. 1, e.g., into the body. Analog-to-digital conversion needs to take place at a rate high enough to preserve the characteristics of the reflected echo signals from the object, particularly if it is moving. As one non-limiting example, with an ultrasound signal centered at 5 MHz, analog-to-digital (A-to-D) conversion rates should be 20 MHz or higher for moving objects in the blood stream. The A-to-D converter must also have sufficient accuracy to preserve amplitude information.

The digitized echo outputs are passed to the data processor 22 for subsequent signal processing and stored in the memory 24. The data processor 22 analyzes the electronic echo signals to classify each object. If desired, the results of the object classification may be displayed or used to produce audible tones, alarms, pre-recorded voice messages, or other signals.

FIG. 2 illustrates a flowchart labeled “Classification” that outlines non-limiting, example signal processing procedures that may be performed on an ultrasound echo signal to classify the object that produced the echo. Multiple echo signals may also be used for the classification, but multiple echoes are not required. The broadband ultrasound transducer 14 transmits a broadband ultrasound pulse towards the object 18 (step S1) and detects an associated ultra sound echo of that pulse from the object 18 (step S2). The ultrasound receiver 26 receives the detected echo signal via the broadband transducer 14. The data processor 22, coupled to the ultrasound receiver, analyzes a time duration parameter and a frequency parameter of the detected echo signal (step S3), and classifies the object as a solid or liquid or as gaseous based on the time duration parameter and the frequency parameter of the detected echo signal (step S4). For example, the object may be classified as a solid or liquid when the time duration parameter exceeds a predetermined time duration value and the frequency parameter exceeds a predetermined frequency value. Otherwise, the object is classified as gaseous.

The data processor 22 may perform these functions under the control of a suitable classification program stored in the memory 24. In a preferred but non-limiting embodiment, the data processor 22, rather that using a Doppler based classification methodology, uses a statistical classification algorithm to determine a classification threshold based on the frequency and time duration parameters of the detected echo signal. The statistical classification algorithm includes, for example, a logistic regression that combines the frequency and time duration parameters of the detected echo signal. Higher values of the frequency and time duration parameters produce a statistical result indicating a higher probability that the object is a solid or liquid rather than gaseous. Other statistical analyses could include other methods, such as but not limited to discriminant analysis, recursive partitioning, etc.

In another example embodiment, the data processor 22 also analyzes an amplitude parameter and/or a phase parameter of the detected echo signal. The object may then be classified as a solid or liquid or as gaseous based on the time duration parameter, the frequency parameter, and one or both of the amplitude parameter and the phase parameter of the detected echo signal.

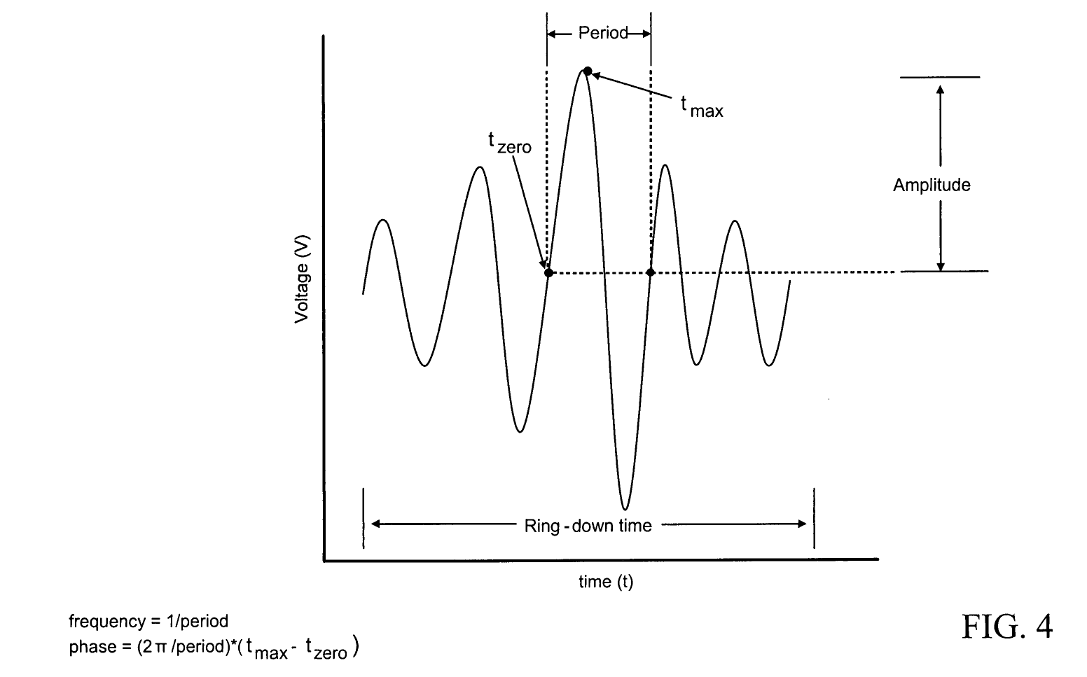

FIG. 4 illustrates multiple echo features that are analyzed by the data processor 22. The time period between t_{zero} and tₘₐₓ may be used to determine the frequency parameter and the phase parameter of the echo. The frequency of the echo is the inverse of the time for one cycle of the signal, and the phase of the signal is the distance in radians that the signal at tₘₐₓ is from the start of a cycle at t_{zero}. Another parameter is the time duration parameter of the echo identified as a ring-down time, where the ring-down time of the echo corresponds to time that the echo signal level exceeds a pre-determined noise threshold. It is desirable for the broadband transducer to have a short ring-down time so that it does not interfere with accurately detecting the echo signal. A fourth echo parameter is the amplitude of the echo. The two most important parameters are the frequency and time duration parameters of the echo, though more than these two parameters may be used.

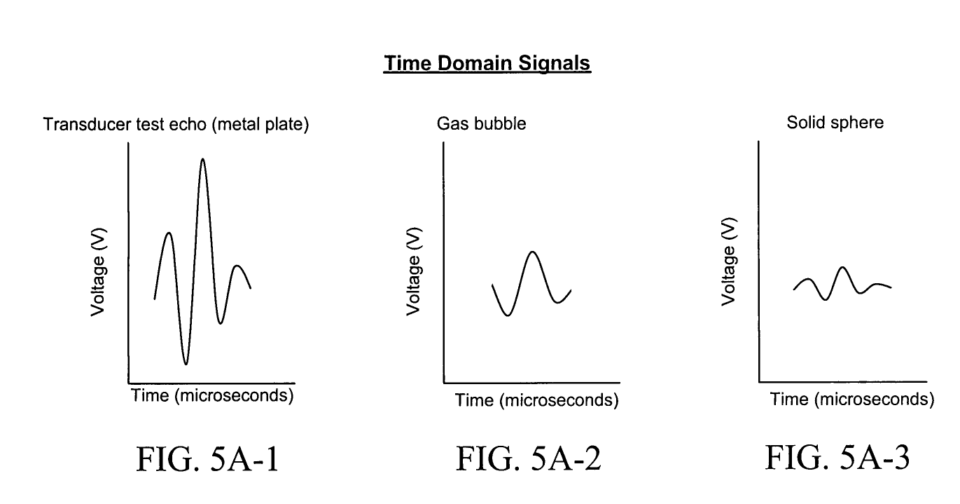

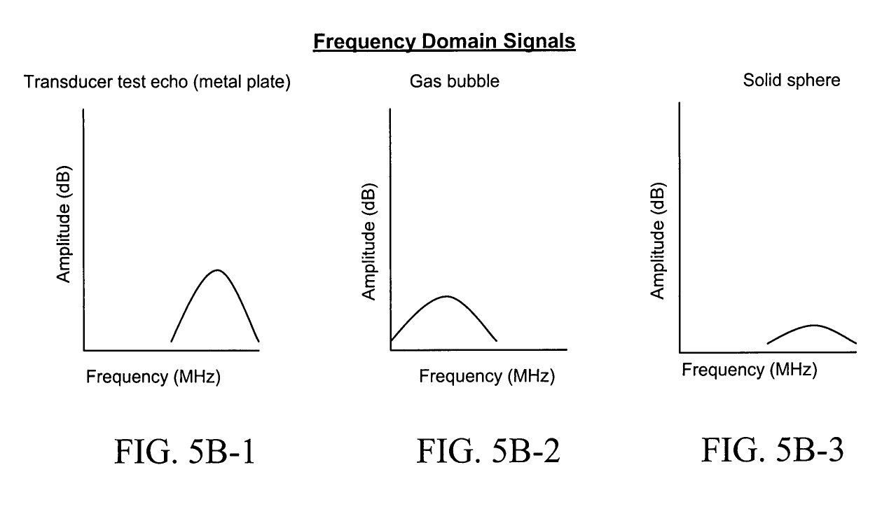

FIG. 5A shows three ultrasonic echo signals in the time domain. The first is a transducer test echo waveform from metal plate; the second is an echo waveform detected for a gas bubble object, e.g., an air bubble; and the third is an echo waveform detected for a solid sphere object, e.g., a glass sphere. Those three signals are transformed into the frequency domain in FIG. 5B and reveal different waveforms with different waveform parameters/characteristics that can be used to classify an object as gaseous (e.g., lower center frequency and medium amplitude) or non-gaseous (e.g., higher center frequency and lower amplitude).

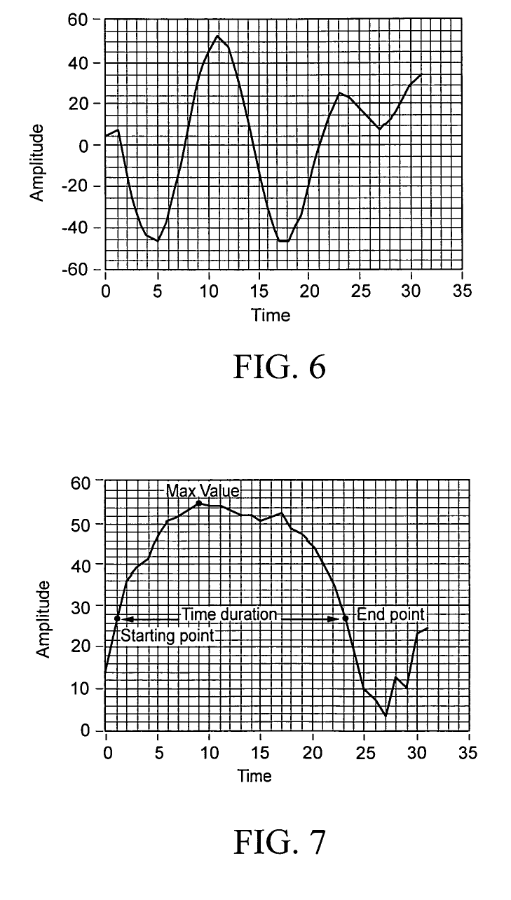

In a preferred example embodiment, these parameters are classified into one of two or more groups established using a statistical classification algorithm derived from a training set of known echo waveforms like the test echoes shown in FIGS. 5A and 5B. Gaseous/non-gaseous classification has been demonstrated using a broadband ultrasound system designed specifically to preferentially enhance signals from moving objects in a fluid. This system, called the EDAC® (emboli detection and classification), extracts a radio frequency (RF) (i.e., ultrasonic) echo signature from each moving particle or embolus, as it passes through a sample volume in the fluid as shown in the example waveform in FIG. 6.

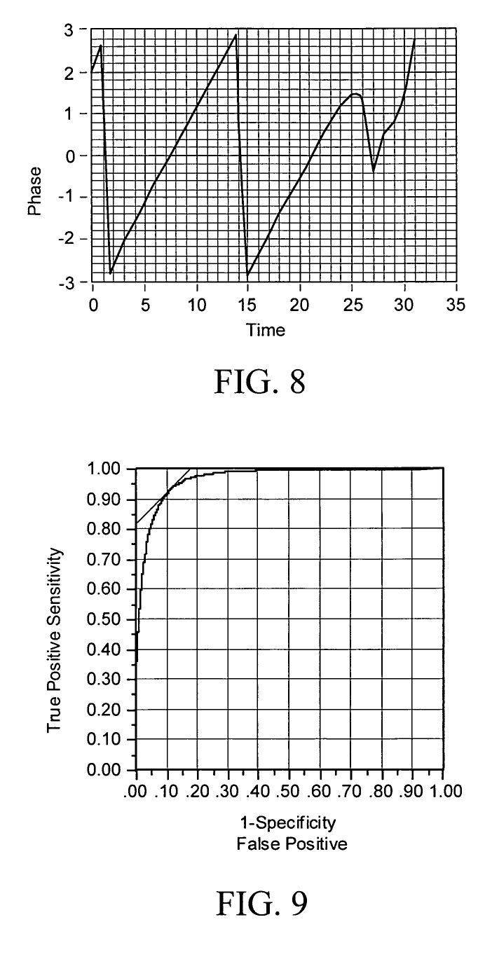

Each RF echo signature is processed to determine the time duration and frequency parameters/characteristics of the signature. Those determinations may be performed in any suitable way, and the techniques described below are non-limiting example techniques. First, the time duration parameter is determined using the Hilbert transform of the echo signature. That Hilbert transform shifts the echo signal 90 degrees in phase, which when combined with the original echo signal shown in FIG. 6, forms an envelope shown in FIG. 7 and the echo's phase signal shown in FIG. 8. The envelope signal in FIG. 7 provides an outline of the signal amplitude without regard to polarity, facilitating the calculation of the time duration parameter. To calculate time duration, the maximum value of the envelope is first found. Then, two points on the envelope before and after the envelope maximum are detected where the envelope magnitude falls below a set threshold (50% of the envelope maximum). The time from this starting point where the envelope first exceeds this threshold to the ending point where the envelope falls below this threshold corresponds to the time duration parameter.

The frequency parameter of the echo signal may then be determined by finding the average change in the phase of the echo signal shown in FIG. 8 over the time duration of the echo signal that was just determined. This is done by first calculating the instantaneous frequency f[t] at each time point in the phase signal φ[t] using a finite difference equation such as f[t]=½(φ[t−1]+φ[t+1]). The average frequency over the interval is then calculated using f_{avg}=sum(f[t])/T, where T is the total number of discretely sampled time points over the time duration of the echo signal. After extracting the time duration and frequency parameters/characteristics of the echo signature in FIG. 6, that echo signature is classified as either gaseous or non-gaseous using logistic regression. Logistic regression is a statistical technique closely related to the least squares multiple regression. In multiple regression, a predicting equation is estimated based on measured values or predictor variables. The predicting equation relates a response variable (Y) to several predictor variables (X) using an equation of the form:

Y=b₀+b₁*X₁+b₂*X₂+ . . . +bₙ*Xₙ [1]

where the b's are estimated coefficients. In a usual multiple regression, the response variable Y has to be a quantitative (numeric) variable.

Logistic regression is an extension of multiple regression in which the response variable is categorical instead of quantitative. It treats the response as either a “0” corresponding to non-gaseous or a “1” corresponding to gaseous and estimates the probability that the RF echo signature falls into one of these two categories based on the value of the predictor variables. To do this, multiple regression is modified to predict probabilities only between 0 and 1 (the usual multiple regression does not have this restriction) and to give equal variance across the response levels. The modification expresses the response using the logit transformation corresponding to:

log(P(gas)/(1−P(gas)) [2]

where P (gas) is the proportion or probability of an emboli being gaseous. Applying the logit transformation [2] to the linear multiple regression formula [1] gives the following equation:

log(P(air)/(1−P(air))=b₀+b₁*X₁+b₂*X₂+ . . . bₙ*Xₙ [3]

Although the two parameters, time duration and frequency, are determined, second order terms formed by the product of time duration and frequency with themselves and each other may also be included in the logistic regression to produce a full quadratic fit. The full quadratic fit incorporates interactions between two values that is modeled in a simple linear fit. The right-hand side of equation 3 is therefore expressed as follows:

a−b₀+b₁*w+b₂*f+b₃*w*f+b₄*w²+b₅*f² [4]

where “w” is the time duration parameter of the echo signature and “f” is the frequency parameter. The second order terms in equation [4] are w*f, w² and f². Substituting “a” into equation [3] and re-arranging yields the function.

P(gas)=1/(1+exp^{-a}). [5]

Equation [5] is fit to the data using statistical software which gives estimates of the values of the b coefficients using mathematic algorithms that seek to find the values of b that produces an equation that fits the measured values with the least total error. Once the b coefficients are obtained for a large set of measured values for different solids and gases in a test environment representative of the actual test conditions (“training data set”), the classification and composition (i.e., density) of individual objects detected in the actual test conditions (“test data set”) may be determined using Equation [5].

Very satisfactory classification results have been obtained using the time duration and frequency parameters of the echo signal. Amplitude may also be useful when the size of the object is known, as gases scatter ultrasound more strongly than solids. For objects whose dimensions are greater than or equal to the wavelength of the incident ultrasound wave, phase shifts may be a good indication of the density of the object relative to the background medium.

A test of an example implementation of the technology is now described. The ultrasonic processing apparatus used was the EDAC® which obtained data from six test runs in which various non-gaseous particles made of olive oil, plastic microbeads, caviar, blood meal were inserted into Tygon tubing and de-aired. The tubing was then placed in a roller pump head and ultrasonically processed using the EDAC®. Additional test runs were performed with air bubbles injected into the tubing.

After randomizing the measured parameters from each RF echo to eliminate bias due to the fact that echoes from gases and solids were acquired in clusters, half the RF echo signatures obtained from this data set were used as a training data set to obtain the b coefficients in Equation [4] and the other half were used as a test data set. This data was fit to a receiver operator characteristic (ROC) curve to determine the sensitivity and specificity of the object classifications. The ROC curve for the test data set is shown in FIG. 9, with sensitivity and specificity values for detecting gases in both the training and test data sets provided in the following table. Sensitivity is related to the rate of true positive detections for a gas. In this example, sensitivity is the percentage of time the classifier equation predicted an object was a gas when it was actually a gas. Selectivity is related to the rate of true negative detections. In this example, selectivity is the percentage of time that the classification prediction predicted an object was a solid when it was actually a solid:

| Data Set | Sensitivity | Selectivity |
|---|---|---|
| Training | 91.7% | 86.1% |
| Test | 91.9% | 86.3% |

These values show excellent performance of the classification algorithm for both stationary and moving objects. The excellent classification for moving objects is particularly significant given that the Doppler-based object detection techniques described in the Background do not perform as well for moving objects. For example, transcranial Doppler ultrasound is highly operator-dependent and more susceptible to noise artifacts. In addition, the use of signal polarity outlined by Hileman only applied to objects greater than or equal to the wavelength of the ultrasound wave. The use of signal amplitude as described by Hileman requires prior knowledge of the object's size (not required with the present technology) because echo amplitude depends on both the composition and size of an object.

Accordingly, the problems identified in the Background are overcome by using multiple features of a broadband ultrasound echo signal including at least pulse duration and frequency to predict whether the signal is produced by a solid or a gaseous embolus. In the preferred example embodiment, multiple echo features are combined into a statistical discriminant analysis to determine an optimal fitting function for those features. Modeling, simulation, testing and mathematical analysis were used to determine parameters for classifying echo signals.

The technology is effective for classifying both stationary objects and moving objects. In one medical application, the object may be an embolus in a blood stream, and the embolus may be classified as a gas bubble, a clot, or a solid particle. Moreover, the technology has other useful applications such as determining a density of an object. Specific non-limiting example applications include: monitoring emboli during extracorporeal bypass procedures, monitoring emboli in-vivo during surgical procures, decompression sickness studies, other cases where emboli are known to be generated in-vivo, and detecting the presence of entrained air and other particles in a fluid system in industrial systems.

Although various example embodiments have been shown and described in detail, the claims are not limited to any particular embodiment or example. None of the above description should be read as implying that any particular element, step, range, or function is essential such that it must be included in the claims scope. Reference to an element in the singular is not intended to mean “one and only one” unless explicitly so stated, but rather “one or more.” The scope of patented subject matter is defined only by the claims. The extent of legal protection is defined by the words recited in the allowed claims and their equivalents. All structural and functional equivalents to the elements of the above-described example embodiments that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. No claim is intended to invoke paragraph 6 of 35 USC §112 unless the words “means for” or “step for” are used. Furthermore, no feature, component, or step in the present disclosure is intended to be dedicated to the public regardless of whether the feature, component, or step is explicitly recited in the claims.

1. An ultrasonic pulse echo apparatus for classifying an object, comprising:
a broadband ultrasound transducer for transmitting a broadband ultrasound pulse towards the object and detecting an associated ultra sound echo of that pulse from the object;
an ultrasound receiver for receiving the detected echo signal; and
a signal processor, coupled to the ultrasound receiver, configured to analyze a time duration parameter and a frequency parameter of the detected echo signal and classify the object as a solid or liquid or as gaseous based on the time duration parameter and the frequency parameter of the detected echo signal.

2. The ultrasonic pulse echo apparatus in claim 1, wherein the signal processor is configured to classify the object as a solid or liquid when the time duration parameter exceeds a predetermined time duration value and the frequency parameter exceeds a predetermined frequency value, and otherwise, to classify the object as gaseous.

3. The ultrasonic pulse echo apparatus in claim 1, wherein the signal processor is configured to use a statistical classification algorithm to determine a classification threshold based on the frequency and time duration parameters of the detected echo signal.

4. The ultrasonic pulse echo apparatus in claim 3, wherein the statistical classification algorithm includes a logistic regression that combines the frequency and time duration parameters of the detected echo signal, wherein higher values of the frequency and time duration parameters produce a statistical result indicating a higher probability that the object is a solid or liquid rather than gaseous.

5. The ultrasonic pulse echo apparatus in claim 1, the signal processor is configured to analyze an amplitude parameter and/or a phase parameter of the detected echo signal and classify the object as a solid or liquid or as gaseous based on the time duration parameter, the frequency parameter, and one or both of the amplitude parameter and the phase parameter of the detected echo signal.

6. The ultrasonic pulse echo apparatus in claim 1, wherein the object is moving when the ultrasound echo from the object is detected.

7. The ultrasonic pulse echo apparatus in claim 1, wherein the broadband transducer has a percent bandwidth of at least 50% of a center frequency of the broadband transducer.

8. The ultrasonic pulse echo apparatus in claim 1, wherein the broadband transducer is a piezoelectric transducer and has a bandwidth frequency range between approximately 1 MHz and 10 MHz.

9. The ultrasonic pulse echo apparatus in claim 1, wherein the signal processor is configured to determine a density of the object based on the time duration parameter and the frequency parameter.

10. The ultrasonic pulse echo apparatus in claim 1, wherein object is an embolus in a blood stream, and wherein the embolus may be classified as a gas bubble, a clot, or a solid particle.

11. A classification method implemented in an ultrasonic pulse echo apparatus, comprising:
transmitting a broadband ultrasound pulse towards the object;
detecting an associated ultrasound echo of that pulse from the object;
analyzing a time duration parameter and a frequency parameter of the detected echo signal; and
classifying the object as a solid or liquid or as gaseous based on the time duration parameter and the frequency parameter of the detected echo signal.

12. The method in claim 11, further comprising:
classifying the object is a solid or liquid when the time duration parameter exceeds a predetermined time duration value and the frequency parameter exceeds a predetermined frequency value, and otherwise, classifying the object as gaseous.

13. The method in claim 11, further comprising:
using a statistical classification algorithm to determine a classification threshold based on the frequency and time duration parameters of the detected echo signal.

14. The method in claim 13, wherein the statistical classification algorithm includes a logistic regression that combines the frequency and time duration parameters of the detected echo signal, wherein higher values of the frequency and time duration parameters produce a statistical result indicating a higher probability that the object is a solid or liquid rather than gaseous.

15. The method in claim 11, wherein the object is moving when the ultra sound echo from the object is detected.

16. The method in claim 11, further comprising:
determining a density of the object based on the time duration parameter and the frequency parameter.

17. The method in claim 11, wherein object is an embolus in a blood stream, and wherein the embolus may be classified as a gas bubble, a clot, or a solid particle.

18. The method in claim 11, further comprising:
analyzing an amplitude parameter and/or a phase parameter of the detected echo signal, and
classifying the object as a solid or liquid or as gaseous based on the time duration parameter, the frequency parameter, and one or both of the amplitude parameter and the phase parameter of the detected echo signal.

19. A computer program product comprising a computer program embodied in a computer-readable medium, wherein the computer program, when executed by a computer, causes the computer to perform the following steps:
receiving for analysis a signal associated with an ultrasound echo of an ultrasound pulse reflected from an object to be classified;
analyzing a time duration parameter and a frequency parameter of the ultrasound echo signal; and
classifying the object as a solid or liquid or as gaseous based on the time duration parameter and the frequency parameter of the detected ultrasound echo signal.

20. The computer program product in claim 19, wherein the computer program, when executed by the computer, causes the computer to perform the following step:
classifying the object as a solid or liquid when the time duration parameter exceeds a predetermined time duration value and the frequency parameter exceeds a predetermined frequency value, and otherwise, classifying the object as gaseous.

21. The computer program product in claim 19, wherein the computer program, when executed by the computer, causes the computer to perform the following step:
using a statistical classification algorithm to determine a classification threshold based on the frequency and time duration parameters of the detected echo signal.

22. The computer program product in claim 19, wherein the computer program, when executed by the computer, causes the computer to perform the following step:
using a statistical classification algorithm to determine a classification threshold based on the frequency and time duration parameters of the detected echo signal.

23. The computer program product in claim 19, wherein the statistical classification algorithm includes a logistic regression that combines the frequency and time duration parameters of the detected echo signal, wherein higher values of the frequency and time duration parameters produce a statistical result indicating a higher probability that the object is a solid or liquid rather than gaseous.

24. The computer program product in claim 19, wherein the object is moving when the ultra sound echo from the object is detected.
